(19) United States
(12) Patent Application Publication
(10) Pub. No.: US 2025/0325340 A1
(43) Pub. Date: Oct. 23, 2025

(54) MODULAR HOSPITAL ELECTROENCEPHALOGRAM (EEG) CART

(71) Applicants: Mary HINTZE, Phoenix, AZ (US); Andrew DEROS, Tempe, AZ (US); Tyler SMITH, Phoenix, AZ (US); Uriel ORNELAS, Glendale, AZ (US)

(72) Inventors: Mary HINTZE, Phoenix, AZ (US); Andrew DEROS, Tempe, AZ (US); Tyler SMITH, Phoenix, AZ (US); Uriel ORNELAS, Glendale, AZ (US)

(73) Assignee: Arizona Board of Regents on Behalf of Arizona State University, Scottsdale, AZ (US)

(21) Appl. No.: 19/183,491

(22) Filed: Apr. 18, 2025

Related U.S. Application Data
(60) Provisional application No. 63/636,668, filed on Apr. 19, 2024.

(51) Int. Cl. A61B 50/13; A61B 5/386; A61B 50/33; B62B 5/06

(52) U.S. Cl. CPC A61B 50/13; A61B 5/386; A61B 50/33; A61B 2560/0437; A61B 2560/045; A61B 2562/182; B62B 5/06

(57) ABSTRACT

A modular electroencephalogram (EEG) cart designed for hospital use, characterized by its versatility and ease of use is disclosed. The EEG cart features a wheeled frame with a top surface to aid technicians during procedures. A detachable technician panel, equipped with multiple drawers and a keyboard tray, is oriented for convenient access by the technician. Alongside, a cable recess panel with numerous hooks allows for efficient cable management and ease of cart transport. On the opposite side, an IT panel is installed, providing access to internal compartment for maintenance and IT serviceability. Facing the patient, a patient panel with pull-out trays readily available to hold medical equipment necessary for patient treatment. An upward-extending camera neck designed to securely hold both a monitor and a camera, for monitoring EEG procedures is also typically included. The modular cart streamlines EEG operations with its ergonomic design and functional features.

### RELATED APPLICATIONS

This application claims the benefit of U.S. provisional patent application 63/636,668, filed Apr. 19, 2024, to Hintze et al., titled “Modular Hospital Electroencephalogram (EEG) Cart,” the entirety of the disclosure of which is hereby incorporated into this disclosure by reference thereto.

### TECHNICAL FIELD

This document relates to a hospital electroencephalogram (EEG) cart used to transport EEG equipment.

### BACKGROUND

Hospital electroencephalogram (EEG) carts are used to provide medical equipment for neurology and neurodiagnostics. EEG is a crucial tool for monitoring and recording electrical brain activity, aiding in diagnosing and treating various neurological conditions such as epilepsy, sleep disorders, and brain injuries. Over the years, EEG technology has advanced significantly, becoming sophisticated and capable of providing detailed insights into brain activity. These advancements require equipment that supports the latest EEG systems and components. In addition, neurodiagnostics is continually evolving, and new components and technologies are introduced regularly.

### SUMMARY

In some embodiments, a modular electroencephalogram (EEG) cart includes a frame mounted on a plurality of wheels. A top surface may be mounted to the frame. In some embodiments, the top surface is configured to provide a surface to a technician using the modular EEG cart. In some embodiments, the modular EEG cart includes a technician panel removably mounted to the frame and configured to face the technician during use. The technician panel may have a plurality of drawers and a keyboard tray. The modular EEG cart may include a cable recess panel removably mounted to the frame adjacent to the technician panel, the cable recess panel may include a plurality of cable hooks configured to support a cable during transport of the EEG cart. In some embodiments, the modular EEG cart includes an IT panel removably mounted to the frame opposite the cable recess panel, the IT panel configured to provide access to an interior compartment of the modular EEG cart. A patient panel may also be removably mounted to the frame and configured to face a patient during use. The patient panel may include a plurality of pull-out trays configured to support medical equipment for use in treating the patient.

In some embodiments, a photic arm is mounted to the frame. A neck is mounted to and extends upward from the frame. A camera may be mounted to the neck on a turntable camera mount configured to permit the camera to rotate. In some embodiments, the neck is configured to support a monitor. The neck may be hollow and configured to route cables down through the neck. In some embodiments, the frame includes a base and a plurality of structural support members extending up from the base. The base may be configured to be heavier than the plurality of structural members.

In some embodiments, the technician panel, the cable recess panel, the IT panel, and the patient panel are configured to be removed and replaced without requiring replacement of additional components of the modular EEG cart. The modular EEG cart may include a grounding chain attached to the frame and configured to ground electronics mounted to the frame. The modular EEG cart may have a frame sized to allow entry into a standard patient room, having a door width of not more than 1.0 meter.

In some embodiments, a modular EEG cart includes a frame, a technician panel, a cable recess panel, and an IT panel. Each panel may be removably mounted to the frame. The modular EEG cart may also include a neck extending upward from the frame. A camera may be mounted on the neck. The technician panel may include a drawer. The cable recess panel may include a recess, the recess having hooks configured to support cables during transport of the modular EEG cart. In some embodiments, the IT panel is configured to provide access to an interior compartment of the modular EEG cart. In some embodiments, the modular EEG cart includes a monitor coupled to the neck.

In some embodiments, the IT panel is mounted to the frame opposite the cable recess panel. A base may be mounted to the bottom of the frame. The base may be coupled to a plurality of wheels. A photic arm may be mounted to the frame. A modular EEG cart may include a patient panel removably mounted to the frame opposite the technician panel. The patient panel may have a tray disposed therein, the tray configured to support medical equipment for use in treating a patient. The patient panel may include a power supply connector for supporting medical equipment for treating the patient.

An infrared light source may be mounted to the neck. In some embodiments, a modular EEG cart or components thereof, are constructed from non-porous, smooth materials resistant to staining and able to withstand frequent sanitization procedures. The modular EEG cart may be constructed from medical-grade stainless steel, antimicrobial-coated plastics, or sealed composite materials that permit the use of hospital-grade disinfectants for effective cleaning. A top surface may be mounted to the frame, the top surface may be configured to provide a surface to a technician using the EEG cart. In some embodiments, a modular EEG cart includes a base, a neck extending from the base, the neck having a hinge, and a camera coupled to the neck opposite the base. In some embodiments, the base is mounted on a plurality of wheels.

The modular EEG cart may include a photic arm mounted to the neck. The camera may be mounted to the neck on a turntable camera mount configured to permit the camera to rotate. A modular EEG cart may also include a grounding chain attached to the base and configured to ground electronics mounted to the modular EEG cart.

The foregoing and other aspects, features, and advantages will be apparent from the DESCRIPTION and DRAWINGS, and from the CLAIMS, if any are included.

### BRIEF DESCRIPTION OF THE DRAWINGS

Implementations will hereinafter be described in conjunction with the appended and/or included DRAWINGS, where like designations denote like elements, and:

FIG. 1 shows a front perspective view of a modular EEG cart according to some embodiments.

FIG. 2 shows a rear perspective of the modular EEG cart of FIG. 1.

FIG. 3 shows a front perspective view of a modular EEG cart according to some embodiments.

FIG. 4 shows a rear perspective of the modular EEG cart of FIG. 3.

FIG. 5 shows a rear perspective of a modular EEG cart according to some embodiments.

FIG. 6 shows a front perspective view of a modular EEG cart according to some embodiments.

FIG. 7 shows a rear view of the modular EEG cart of FIG. 6.

FIG. 8 shows a rear perspective of the modular EEG cart of FIG. 6 with panels removed to view interior structures.

FIG. 9 shows a front, side perspective of the modular EEG cart of FIG. 6 with panels removed to view interior structures.

FIG. 10 shows a side view of the modular EEG cart of FIG. 6 with panels removed to view interior structures.

FIG. 11A shows an embodiment of a module EEG cart in a closed configuration

FIG. 11B shows the module EEG cart of FIG. 11A in an open configuration.

FIG. 12A shows an embodiment of a module EEG cart.

FIG. 12B shows an embodiment of a module EEG cart.

FIG. 13A shows a rear, left perspective view of an embodiment of a module EEG cart in an open configuration.

FIG. 13B shows a front, right perspective view of the module EEG cart of FIG. 13A in a closed configuration.

FIG. 14A shows a rear, left perspective view of an embodiment of a module EEG cart in an open configuration.

FIG. 14B shows a rear, left perspective view of the module EEG cart of FIG. 14A in a closed configuration.

FIG. 14C shows a front, right perspective view of the module EEG cart of FIG. 14B.

FIG. 15A shows a front, left perspective view of an embodiment of a module EEG cart in an open configuration.

FIG. 15B shows a front, left perspective view of a chair for use with the module EEG cart of 15A, according to some embodiments.

FIG. 15C shows a front, left perspective view of the module EEG cart of FIG. 15A in the closed configuration in a cubicle of FIG. 15B.

FIG. 15D shows a rear, right perspective view of the module EEG cart of FIG. 15A in the closed configuration in a cubicle of FIG. 15B.

### DETAILED DESCRIPTION

The following detailed description provides numerous specific details. Those skilled in the relevant arts understand that embodiments of the disclosure may be practiced without these specific details. The disclosure may also be practiced in different and alternative configurations.

Unless specifically noted, it is intended that the words and phrases in the specification and the claims be given their plain, ordinary, and accustomed meaning to those of ordinary skill in the applicable arts. The singular forms “a,” “an,” and “the” include plural referents unless the context clearly dictates otherwise. Thus, for example, a reference to “a step” includes a reference to one or more of such steps. The words “exemplary,” “example,” “embodiment,” or various forms thereof are used herein to mean serving as an example, instance, or illustration. Any aspect or feature described herein as “exemplary” or as an “example” is not necessarily to be construed as preferred or advantageous over other aspects or designs. The examples are provided solely for purposes of clarity and understanding and do not limit or restrict the disclosure. It is to be appreciated that a myriad of additional or alternate examples of varying scope could have been presented, but have been omitted for purposes of brevity.

Throughout the description and claims of this specification, the words “comprise” and “contain” and variations of the words, for example “comprising” and “comprises”, mean “including but not limited to”, and are not intended to (and do not) exclude other components.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one.

The present disclosure relates to modular electroencephalogram (EEG) carts. Modular EEG carts enable the transport of medical equipment and technology, particularly within the fields of neurology and neurodiagnostics. The modular EEG carts disclosed herein are versatile and adaptable solutions designed to enhance EEG testing in medical facilities. Featuring a modular design, the modular EEG carts disclosed herein accommodate evolving EEG technology, ensuring compatibility with the latest equipment.

EEG testing can be time-consuming and require the presence of skilled technicians. To streamline the testing process and increase efficiency, the modular EEG cart disclosed herein features several enhancements designed to minimize technician workload and enhance workflow. Improved efficiency improves patient experience and reduces costs. The modular EEG carts disclosed herein aim to provide a comprehensive solution that not only supports the latest EEG technology but also prioritizes hygiene, patient comfort, technician efficiency, and adaptability to future developments, ultimately improving the quality of EEG testing in hospital settings. The cleanable design materials and layout prioritize hygiene in healthcare settings, reducing the risk of cross-contamination and ensuring a more sanitary environment. The present disclosure incorporates a sleek, cleanable design that prioritizes patient and technician comfort.

Like all medical fields, neurodiagnostics is continually evolving. New components and technologies are regularly introduced. The modular EEG cart disclosed herein allows providers to easily adapt to these changes and stay current with the latest EEG equipment. Unlike traditional EEG carts that may become obsolete as technology advances, the modular design disclosed herein allows for easy customization and adaptability to accommodate evolving EEG technology. The adaptability of the cart stems from the fact that individual components of the modular hospital EEG cart can be redesigned and replaced without requiring that the entire cart be replaced.

The modular EEG cart may also have a technical panel, allowing a technician to access its internal components. This reduces downtime and service costs. The modular EEG cart offers numerous innovations, including a concealed cable management system, adjustable monitor mount, lockable drawers for technician materials, and a cleanable, user-friendly design that prioritizes patient comfort and hygiene. Additional features include lockable drawers, a pull-out keyboard, and cable routing, all aimed at improving the workflow and reducing the technician's workload.

The modular EEG cart disclosed herein also improves technical aspects related to the EEG testing. The modular EEG cart includes a grounding chain and cable management system that improves patient safety and reduces electronic interference during the recording of brain activity. This is critical to accurate EEG recordings.

The modular EEG cart accommodates various EEG testing and recording components. For example, the modular EEG cart may include cameras, photic arms having light sources, microphones, IR light sources, and other features. The cameras and microphone enable comprehensive patient monitoring during EEG tests, capturing behavioral and verbal responses for comprehensive patient monitoring during EEG tests. This data is valuable for clinicians in diagnosing neurological conditions.

In summary, the presently disclosed modular EEG cart's advantages over current EEG cart technology encompass modularity, improved workspace functionality, patient experience, versatility, safety enhancements, reduced interference, ease of maintenance, cost-efficiency, data capture capabilities, and enhanced hygiene. These advantages collectively make it a valuable innovation with the potential to improve EEG testing quality and efficiency while ensuring adaptability to future technological advancements.

With its potential to streamline EEG procedures, improve technician efficiency, and reduce electronic interference, the modular hospital EEG cart presents a valuable opportunity for companies seeking to enhance neurodiagnostic capabilities and patient care within healthcare settings. Its commercial potential lies in improving the overall EEG testing experience and maintaining adaptability to future technological advancements.

Features of the disclosure will be described with reference to the embodiments shown in the Figures. Some features may be discussed with reference to only certain figures. However, unless context demands otherwise, like numbers designating like features throughout the figures.

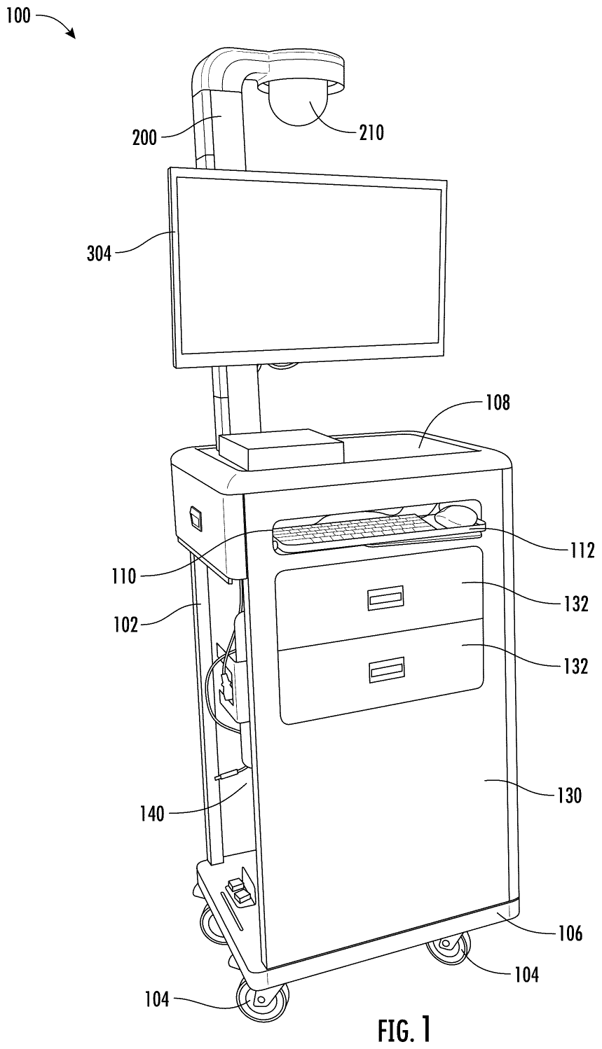

FIG. 1 shows modular EEG cart 100. Modular EEG cart 100 includes frame 102. Frame 102 provides structure to modular EEG cart 100 and is the component upon which most other components are mounted. Frame 102 may be formed of a structurally sound material such as aluminum. A base 106 may be mounted to the bottom of frame 102. In some embodiments, base 106 is formed of a denser material than frame 102. This may make base 106 heavier than frame 102. A heavy base 106 helps modular EEG cart 100 avoid tipping. In some embodiments, frame 102 may be formed of aluminum sheet metal and base 106 may be formed of steel. Casters 104 may be mounted to base 106. In some embodiments, casters 104 are mounted to base 106 or to frame 102. Casters 104 allows modular EEG cart 100 to be wheeled around a hospital. In some embodiments, modular EEG cart 100 includes four casters 104.

In some embodiments, frame 102 may be sized according to hospital standards to ensure it can fit through any doorway. For example, frame 102 may be sized to fit through the tightest areas of the Neonatal Intensive Care Unit (NICU). In some embodiments, frame 102 includes clamps to hold all components stable, for example, the computer components. Clamps may be completely adjustable to meet any sizing constraints or changes to hardware.

FIG. 1 shows technician panel 130 mounted to frame 102. Technician panel 130 is designed to face the technician and away from the patient when in use. Technician panel 130 may be formed from powder-coated aluminum sheet metal. In some embodiments, technician panel 130 includes drawers 132 for the storage of items that the technician needs to use when testing patients. In some embodiments, the technician panel 130 has two drawers 132. Drawers 132 may be located underneath cutout 110 for keyboard tray 112. Keyboard tray 112 and drawers 132 may be mounted to technician panel 130 using drawer slides. This permits drawers 132 and keyboard tray 112 to be smoothly pulled out when in use. Keyboard tray 112 may have a smaller mouse tray attached to the base of the keyboard tray via drawer slides. In some embodiments, technician panel 130's components may be held closed via a releasable fastener. In some implementations, the releasable fastener may be a magnetic latch. Technician panel 130's components may also have a lockable access door that can be opened for the technician to switch on and off any computer components before transportation or for some other purpose. The inclusion of lockable drawers for storing technician materials enhances security and organization. These drawers ensure that essential tools and accessories are readily available while maintaining a clutter-free workspace.

FIG. 1 also shows top surface 108. Top surface 108 may be a custom plastic component with a rubber mat that provides a surface for the technician to set items on the table. Surfaces of modular EEG cart 100 may be configured to be easily cleanable. For example, the outer surfaces may be configured to be easily wiped down. The materials used are easy to clean and maintain, contributing to a more sanitary healthcare environment. Examples of suitable materials include non-porous, smooth materials known for their resistance to staining and ability to withstand frequent sanitization procedures, such as medical-grade stainless steel, antimicrobial-coated plastics, or sealed composite materials that permit the use of hospital-grade disinfectants for effective cleaning.

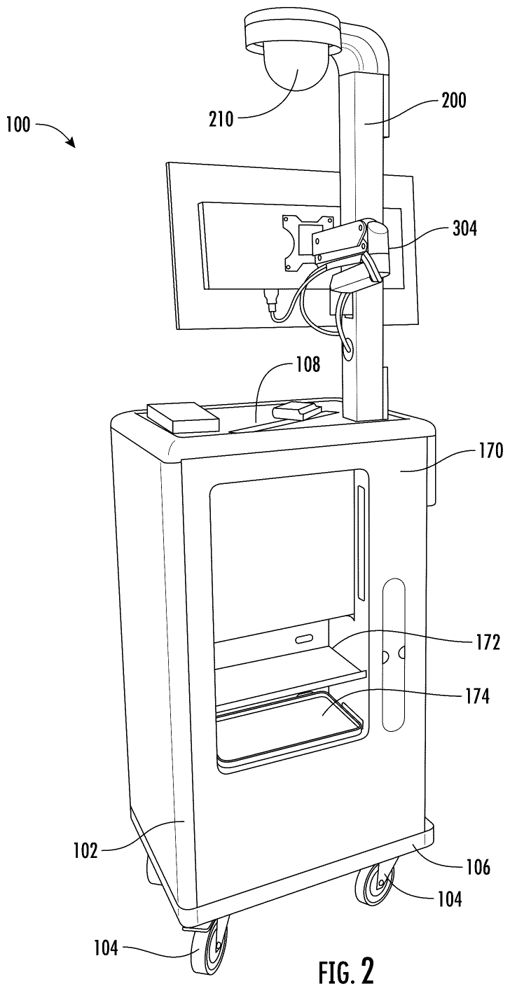

FIG. 2 shows a rear view of modular EEG cart 100, shown in FIG. 1. FIG. 2 shows modular EEG cart 100 having patient panel 170. Patient panel 170 is mounted to frame 102 opposite technician panel 130. Patient panel 170 is configured to face the patient when other systems mounted to modular EEG cart 100 are in use. Like technician panel 130, patient panel 170 may be formed of power-coated aluminum sheet metal. In some embodiments, patient panel 170 may include tray 172 and pull-out tray 174. In some embodiments, each tray 172 and 174 may be lined with rubber to reduce the sliding of patient devices or items that may be placed on trays 172 and 174. In some embodiments, tray 172 and pull-out tray 174 are configured to hold a CPAP machine, an EEG machine, or other items that require direct attachment to the patient. Although FIG. 2 shows one tray 172 and one pull-out tray 174, but other numbers are contemplated. For example, in some embodiments, patient panel 170 may include two pull-out trays 174. Pull-out trays 174 may be mounted on drawer slides and may be held closed with releasable fasteners, such as magnetic latches. The pull-out trays 174 are configured to support medical equipment for use in treating the patient. Patient panel 170 may also have power supplies or other cable attachment to connect and power items that require direct attachment to the patient.

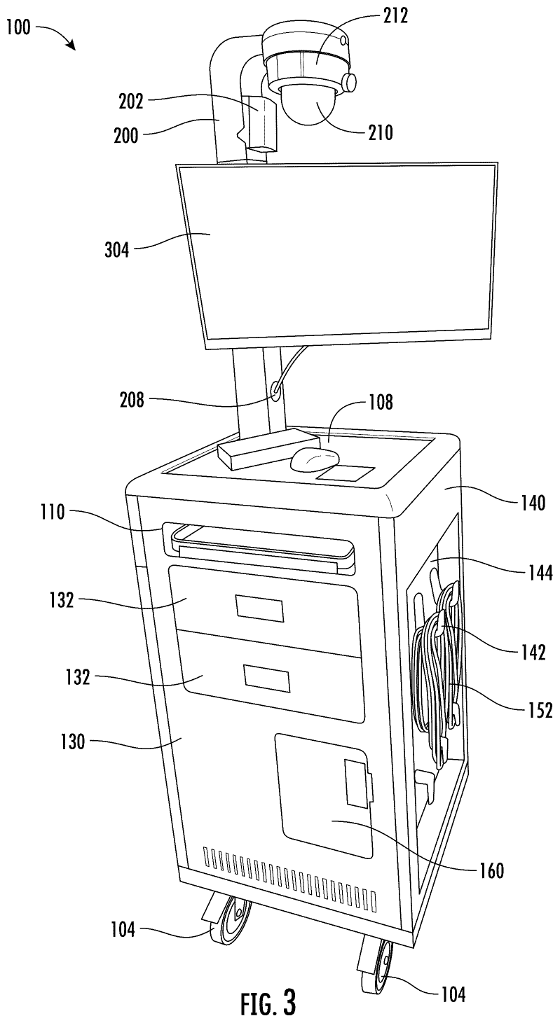

FIG. 3 shows modular EEG cart 100, according to some embodiments. As shown, modular EEG cart 100 includes cable recess panel 140. Cable recess panel 140 may be formed from powder-coated aluminum sheet metal. Cable recess panel 140 may be mounted to frame 102 to make cables 152 accessible to the technician. The cable recess panel 140 may be adjacent to the technician panel 130. As shown in FIG. 3, cable recess panel 140 has recess 144. Cable recess panel 140 may include hooks 142 configured to receive wound cables 152. Hooks 142 that allow for winding up cables to store during transport. In some embodiments, cable recess panel 140 includes three hooks 142. This helps facilitate the secure organization of cables 152 during the transport of modular EEG cart 100. Specifically, the recessed nature minimizes the risk of cables 152 snagging on other objects during transport, resulting in damage to cables 152 or damage to the contacted object.

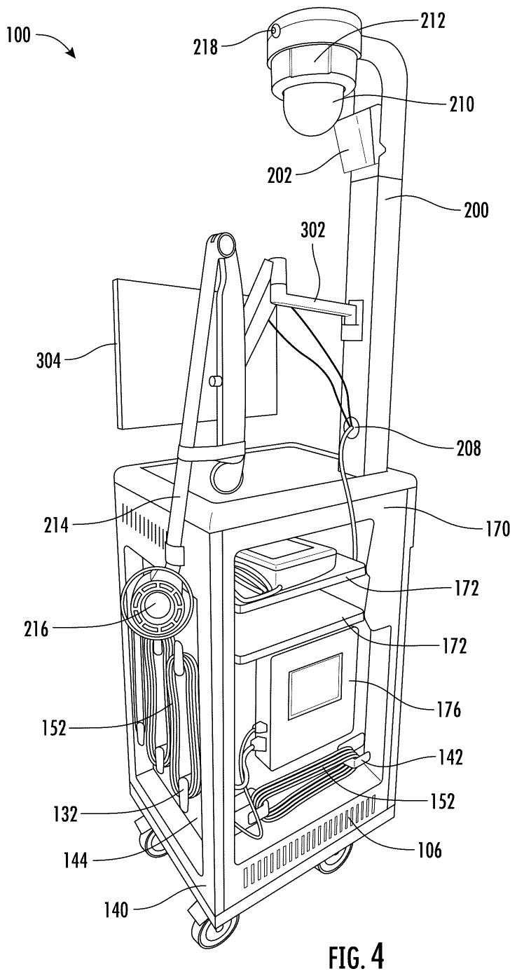

FIG. 4 shows a rear view of modular EEG cart 100 shown in FIG. 3. Patient panel 170 includes a recess for mounting power amplifier 176 via an adjustable mounting plate and an area with hooks 142 for cable management so all items can be easily wrapped and stored when transporting the cart through the hospital.

FIG. 4 also shows neck 200 extending upward from the top of modular EEG cart 100. In some embodiments, neck 200 may be formed from powder-coated aluminum sheet metal. Neck 200 securely mounts cameras, microphones, and IR lights while internally routing cables, reducing clutter and minimizing tripping hazards. To this end, neck 200 may be hollow. Light source 202 and camera 210 may be mounted to neck 200. In some embodiments, neck 200 may have a removable door panel that allows a technician to access the interior of neck 200. This may help the technician organize the cables of the devices coupled to neck 200. A monitor 304 may be mounted to neck 200 with adjustable monitor mount 302. Adjustable monitor mount 302 provides technicians with the ability to customize the monitor's height and viewing angle, optimizing visibility of EEG data and enhancing the testing experience.

Camera 210 may be mounted on turntable camera mount 212. Camera 210 may be mounted with brackets to turntable camera mount 212, which allows camera 210's position to be adjusted through 180 degrees by the technician using a button. In some embodiments, microphone 218 may be mounted to neck 200. All cabling for the devices coupled to neck 200 may run internally down 200 to other portions of modular EEG cart 100. Grommet holes 208 permit cabling to extend through the neck, allowing cabling for the monitor to run seamlessly down the neck into the internal storage cavity. FIG. 4 also shows photic arm 214. Photic arm 214 may be used for photic stimulation of the patient. Photic arm 214 may include a light source 216.

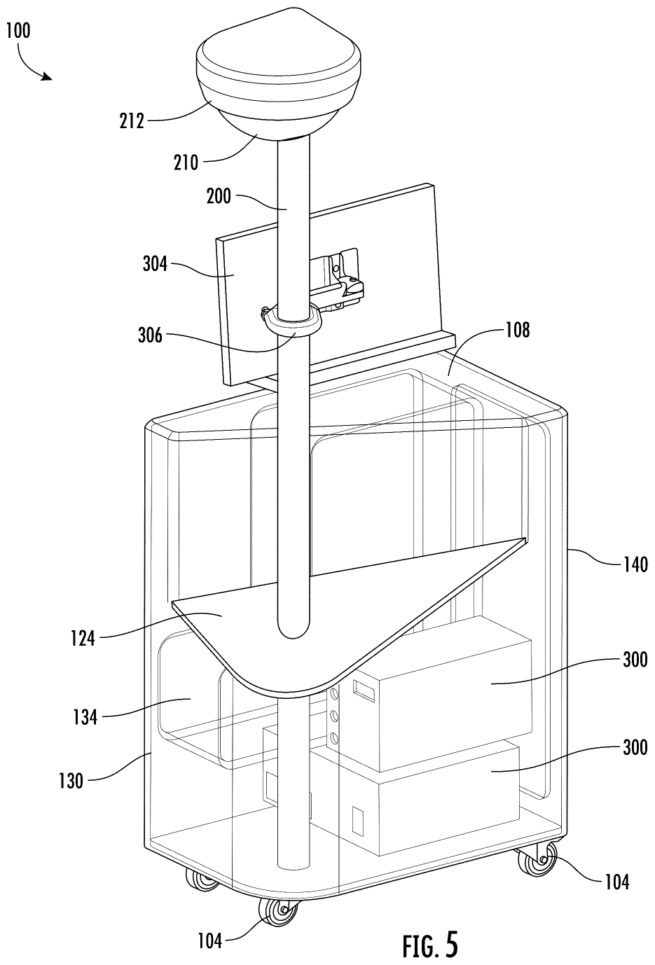

FIG. 5 shows a rear perspective view of modular EEG cart 100 according to some embodiments. The view shown in FIG. 5 is a partially transparent view to permit a better understanding of the structures present in some embodiments of modular EEG cart 100. FIG. 5 shows modular EEG cart 100 on wheels 104. Modular EEG cart 100's interior includes electronics 300. Electronics 300 may be electronics associated with devices coupled to modular EEG cart 100. For example, electronics 300 may include a computer for managing components of modular EEG cart 100. Neck 200 extends from base 106, through panel 124, and above upper surface 108. Neck 200 acts as a support for clamp 306, which supports monitor 304. Camera 210, on turntable camera mount 212, is mounted to the top of neck 200. FIG. 5 also shows cable recess panel 140 and technician panel 130, which includes technical panel compartment 134. The inclusion of a removable panel on the technician side, allowing full access to the internal cavity and cable routing, is a novel design feature. It simplifies maintenance and upgrades, making it easier for technicians to service the cart.

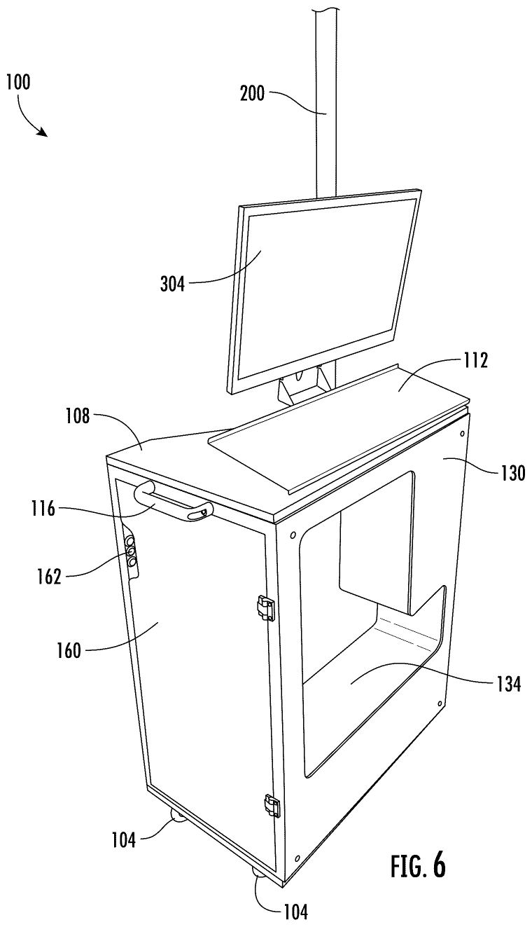

FIG. 6 shows IT panel 160 mounted to frame 102 of modular EEG cart 100. In some embodiments, IT panel 160 is a removable panel or openable panel. IT panel 160 may be removed or opened to allow technicians or IT professionals complete access to all internal components and storage for cable routing and servicing of modular EEG cart 100 or components of modular EEG cart 100. IT panel 160 may be removable or opened with a code or key lock. FIG. 6 shows lock 162, which may be configured to lock IT panel 160. IT panel 160 may also have a handle 116 mounted thereon. Handle 116 may also be mounted on frame 102. Handle 116 may facilitate moving modular EEG cart 100 around the hospital. In some embodiments, modular EEG cart 100 may include a mounting point for a grounding chain to ground electronics mounted frame 102. The grounding chain may implement a press fit PEM and locknut.

The grounding chain and cable management systems reduce electronic interference, ensuring accurate EEG recordings. This is crucial for the reliability of diagnostic results and the effectiveness of treatment plans. The grounding chain integrated into the cart is a novel addition. It plays a crucial role in maintaining electrical grounding, reducing electronic interference during EEG tests, and ensuring the accuracy of recorded brain activity.

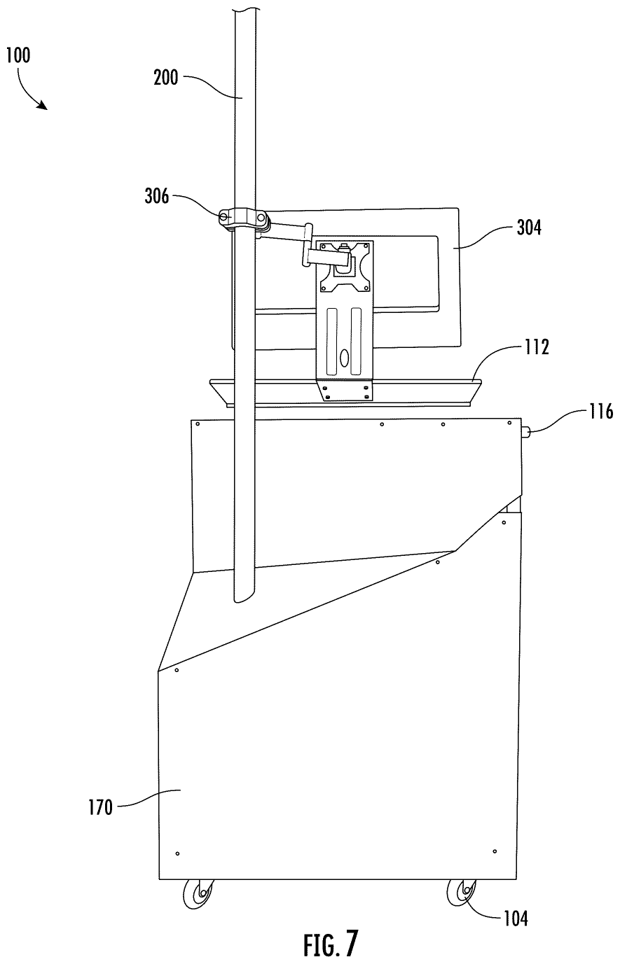

FIG. 7 shows a rear view of the embodiment of a modular EEG cart 100 shown in FIG. 6. Modular EEG cart 100 has a simple patient panel 170 that does not have drawers or features. An embodiment like the one shown in FIG. 7 may be useful in environments where patient connection needs are expected to be minimal or in small environments where smooth designs are preferred. For example, in an intensive care unit, a patient may not need supplemental machines brought to them on modular EEG cart 100 because those supplemental machines are already operating as part of the patient care.

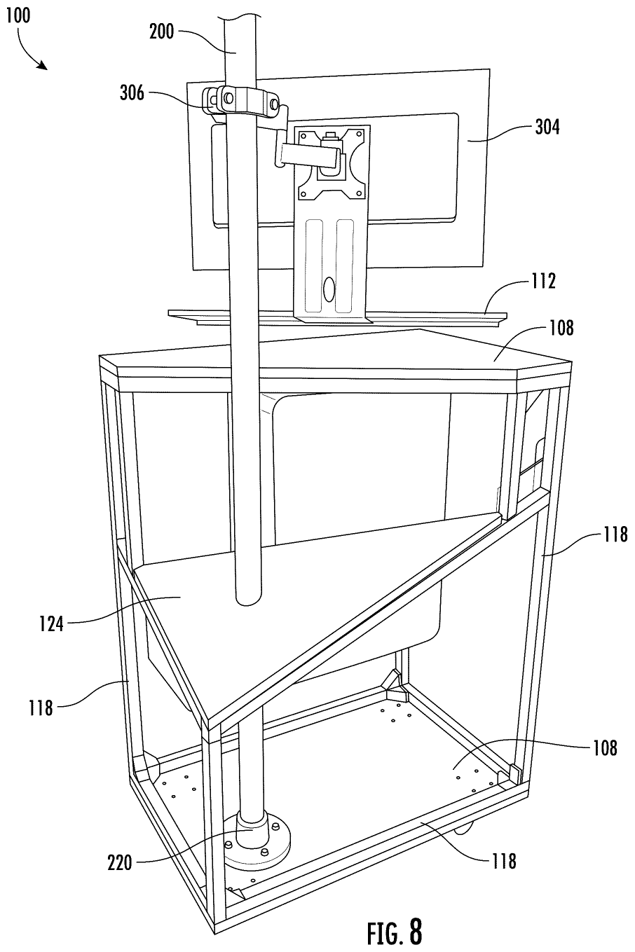

FIG. 8 shows a rear perspective of the modular EEG cart of FIG. 6 with panels removed to view interior structures. Grommet 220 may support neck 200. As shown in FIG. 8, grommet 220 may be coupled to base 106. This provides a strong foundation for neck 200. FIG. 8 also shows structural supports 118. Structural supports 118 are configured to provide structure to modular EEG cart 100.

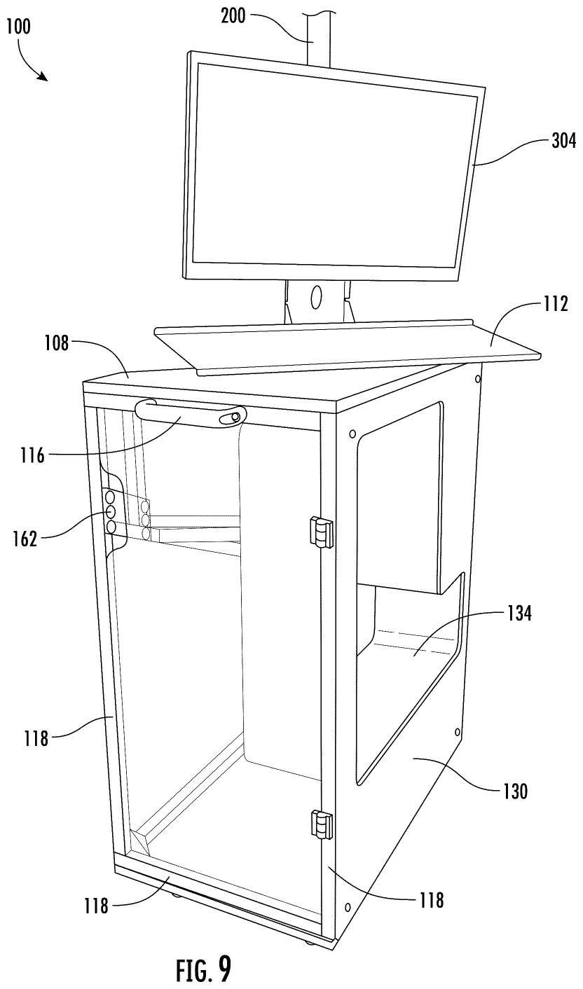

FIG. 9 shows a front, side perspective of the modular EEG cart of FIG. 6 with panels removed to view interior structures. Technician panel 130 has technical panel compartment 134 formed in therein. Technical panel compartment 134 may house other features such as drawers 132 or shelving.

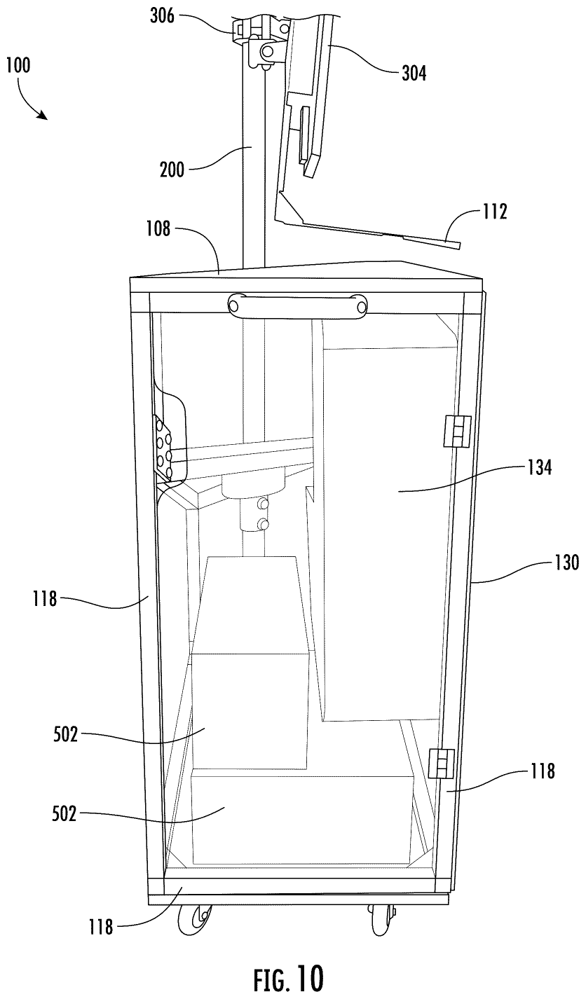

FIG. 10 shows a side view of the modular EEG cart of FIG. 6 with panels removed to view interior structures. Electronic compartments 502 may be configured to hold electronics 300. 502 may be accessible by removing or opening IT panel 160.

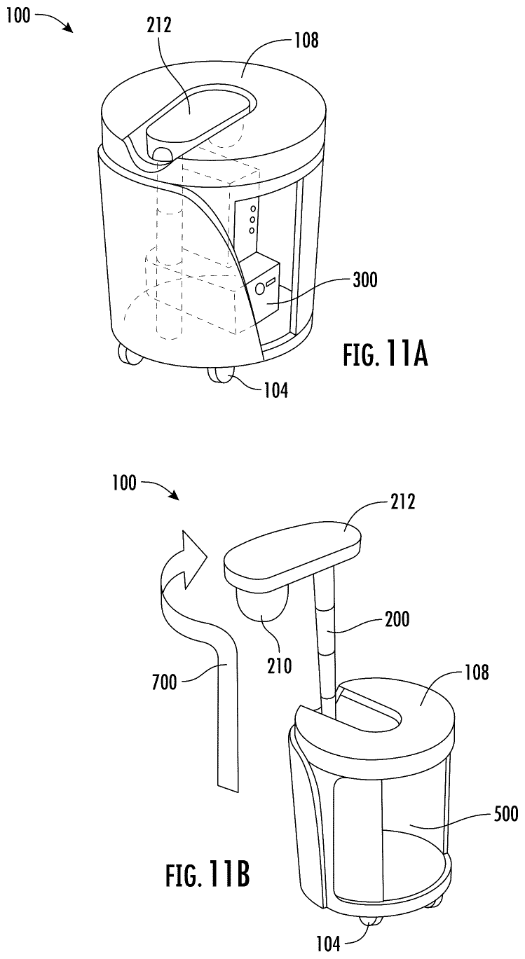

In some embodiments, modular EEG cart 100 may take several forms. Example forms are shown in FIGS. 11A-15D and are described below. The design of modular EEG cart 100 may be driven by aesthetic considerations. For example, patients may become more nervous or anxious during medical procedures. This may result in physiological responses in the patient that impede the collection of accurate data, or may result in a delay in the testing being administered until the patient reduces their anxiety. This is often termed the “white coat” effect.

Good design can reduce the white coat effect and improve patient experiences. One way to reduce patient anxiety is to design machines that do not look like typical, “scary” medical machines.

FIGS. 11A and 11B show an embodiment of modular EEG cart 100 that has a closed configuration, shown in FIG. 11A, and an open configuration, shown in FIG. 11B. This embodiment of modular EEG cart 100 is shaped such that, in the closed configuration, modular EEG cart 100 appears less like a medical cart or medical device and more like a piece of furniture or other stylistic element of a room. This more informal feel makes patients feel more at ease in the medical setting. Although the embodiment shown in FIGS. 11A and 11B is designed with aesthetics in mind, it is no less functional. For example, modular EEG cart 100 may include top surface 108 and electronics 300 stored in compartment 500. Modular EEG cart 100 also has turntable camera mount 212. In this embodiment, turntable camera mount 212 may turn about neck 200. The extension and rotation of camera 210 is indicated with movement arrow 700.

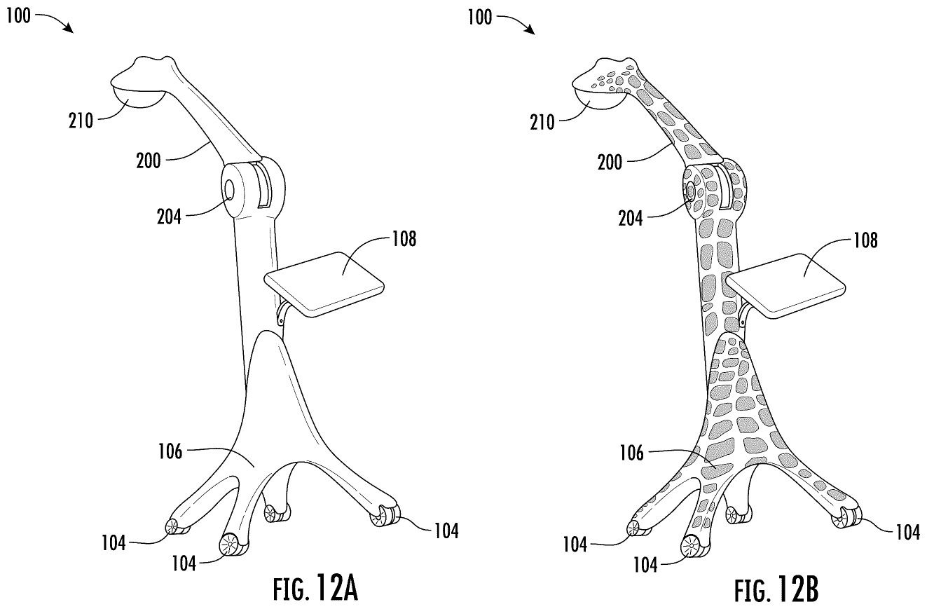

FIGS. 12A and 12B show embodiments of modular EEG cart 100. In the embodiments shown, neck 200 has hinge 204. Hinge 204 may help a technician better position components of modular EEG cart 100. Hinge 204 shown in FIGS. 12A and 12B permit the technician to move camera 210 up or down as needed. Although the embodiments shown in FIGS. 12A and 12B show hinge 204 that moves camera 210 up and down, other hinges 204 may permit camera 210 to move in more directions. The embodiments of modular EEG cart 100 shown in FIGS. 12A and 12B may be particularly useful in a pediatric setting where white coat syndrome may be particularly present. In these embodiments, modular EEG cart 100 is shaped to look more like an animal, for example, a giraffe. Modular EEG cart 100 may also be painted to match the shape modular EEG cart 100 attempts to imitate. FIG. 12B shows modular EEG cart 100 painted to include spots like those that on a giraffe. Like other embodiments, the embodiments shown in FIGS. 12A and 12B include top surface 108 that provides a work surface for technician. Modular EEG cart 100 may also include base 106 with casters 104 to permit modular EEG cart 100 to be rolled to care areas.

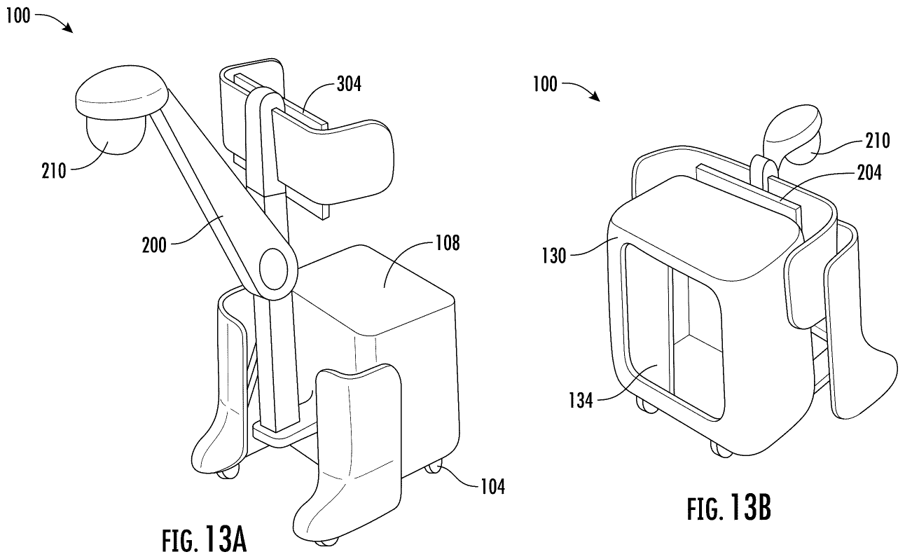

FIG. 13A shows a rear, left perspective view of an embodiment of modular EEG cart 100 in an open configuration and FIG. 13B shows a front, right perspective view of the module EEG cart of FIG. 13A in a closed configuration. The embodiments shown in these figures have a collapsible monitor 304 mount. Like other embodiments of modular EEG cart 100 discussed herein, modular EEG cart 100 is on casters 104 and includes technical panel compartment 134.

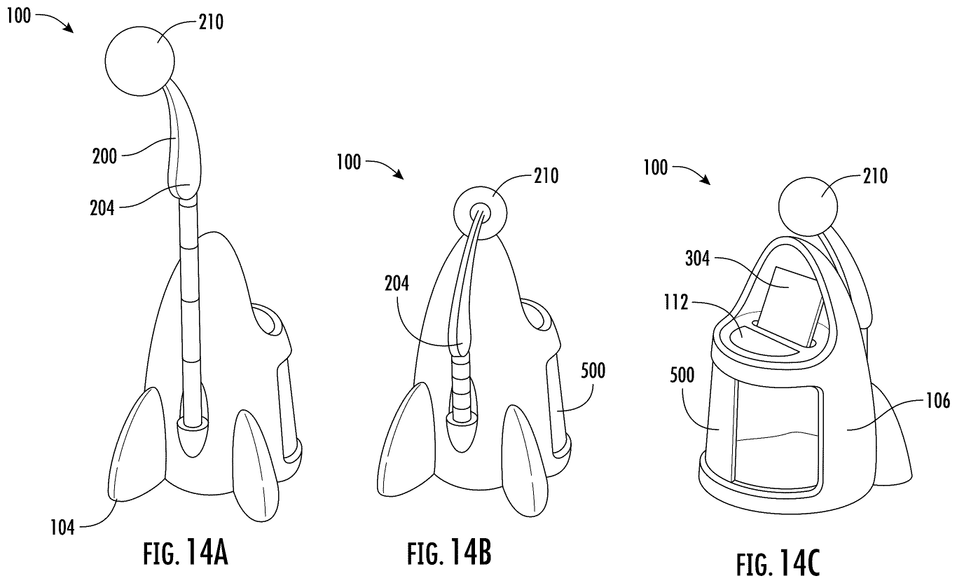

FIGS. 14A-14C show an embodiment of modular EEG cart 100. Modular EEG cart 100 has a telescoping neck 200 that allows the technician to raise camera 210. Neck 200 also has hinge 204 to further allow technician to locate camera 210. Modular EEG cart 100 also includes monitor 304 and keyboard tray 112. Compartment 500 gives the technician space to place items and may also hold electronics 300. In some embodiments, compartment 500 is lockable.

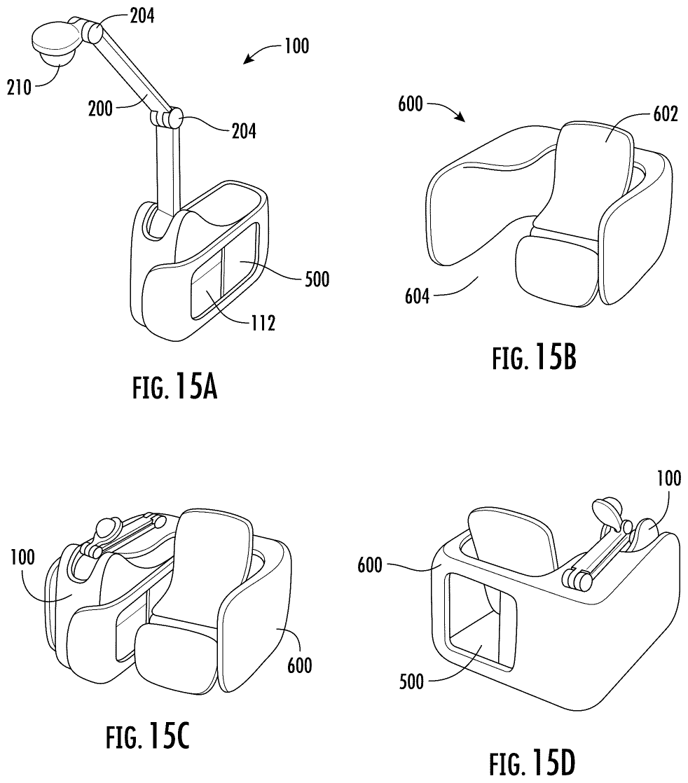

FIGS. 15A-15D show an embodiment of modular EEG cart 100 that is configured to slide into a slot 604 of a module 600. Module 600 may include patient chair 602. FIG. 15A shows modular EEG cart 100 with neck 200. Neck 200 has two hinges 204. Modular EEG cart 100 is sized to fit into slot 604. A patient may rest in patient chair 602 as modular EEG cart 100 is positioned to apply a test to the patient. FIG. 15D shows that module 600 includes compartment 500. In some embodiments, some features of modular EEG cart 100 previously described may located on or coupled to module 600. When a small modular EEG cart 100 is used, moving some components to module 600 may save space. with module 600. FIG. 15A shows a front, left perspective view of an embodiment of a module EEG cart in an open configuration.

It will be understood that implementations of the modular hospital EEG cart include but are not limited to the specific components disclosed herein, as virtually any components consistent with the intended operation of various modular hospital EEG carts may be utilized. Accordingly, for example, it should be understood that, while the drawings and accompanying text show and describe particular modular hospital EEG cart implementations, any such implementation may comprise any shape, size, style, type, model, version, class, grade, measurement, concentration, material, weight, quantity, and/or the like consistent with the intended operation of modular hospital EEG carts.

The concepts disclosed herein are not limited to the specific modular hospital EEG carts shown herein. For example, it is specifically contemplated that the components included in particular modular hospital EEG carts may be formed of any of many different types of materials or combinations that can readily be formed into shaped objects and that are consistent with the intended operation of the modular hospital EEG cart. For example, the components may be formed of: rubbers (synthetic and/or natural) and/or other like materials; glasses (such as fiberglass), carbon-fiber, aramid-fiber, any combination therefore, and/or other like materials; elastomers and/or other like materials; polymers such as thermoplastics (such as ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; plastics and/or other like materials; composites and/or other like materials; metals, such as zinc, magnesium, titanium, copper, iron, steel, carbon steel, alloy steel, tool steel, stainless steel, spring steel, aluminum, and/or other like materials; and/or any combination of the foregoing.

Furthermore, modular hospital EEG carts may be manufactured separately and then assembled together, or any or all of the components may be manufactured simultaneously and integrally joined with one another. Manufacture of these components separately or simultaneously, as understood by those of ordinary skill in the art, may involve 3-D printing, extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular modular hospital EEG cart implementations, it should be readily apparent that a number of modifications may be made without departing from the spirit thereof and that these implementations may be applied to other implementations disclosed or undisclosed. The presently disclosed modular hospital EEG carts are, therefore, to be considered in all respects as illustrative and not restrictive.

What is claimed is:

1. A modular electroencephalogram (EEG) cart, comprising:
a frame mounted on a plurality of wheels and a top surface mounted to the frame, the top surface configured to provide a surface to a technician using the modular EEG cart;
a technician panel removably mounted to the frame and configured to face the technician during use, the technician panel having a plurality of drawers and a keyboard tray;
a cable recess panel removably mounted to the frame adjacent to the technician panel, the cable recess panel having a plurality of cable hooks configured to support a cable during transport of the modular EEG cart;
an IT panel removably mounted to the frame opposite the cable recess panel, the IT panel configured to provide access to an interior compartment of the modular EEG cart;
a patient panel removably mounted to the frame and configured to face a patient during use, the patient panel having a plurality of pull-out trays configured to support medical equipment for use in treating the patient;
a photic arm mounted to the frame;
a neck mounted to and extending upward from the frame; and
a camera mounted to the neck on a turntable camera mount configured to permit the camera to rotate,
wherein the neck is configured to support a monitor,
wherein the neck is hollow and is configured to route cables down through the neck.

2. The modular EEG cart of claim 1, wherein the frame comprises a base and a plurality of structural members extending up from the base, wherein the base is configured to be heavier than the plurality of structural members.

3. The modular EEG cart of claim 1, wherein each of the technician panel, the cable recess panel, the IT panel, and the patient panel is configured to be removed and replaced without requiring replacement of additional components of the modular EEG cart.

4. The modular EEG cart of claim 1, further comprising a grounding chain attached to the frame and configured to ground electronics mounted to the frame.

5. The modular EEG cart of claim 1, wherein the frame is sized to allow entry into a standard patient room, having a door width of not more than 1.0 meter.

6. A modular EEG cart comprising:
a frame;
a technician panel, cable recess panel, and IT panel, each panel removably mounted to the frame;
a neck extending upward from the frame; and
a camera mounted to the neck;
wherein the technician panel includes a drawer,
wherein the cable recess panel comprises a recess, the recess having hooks configured to support cables during transport of the modular EEG cart, and
wherein the IT panel is configured to provide access to an interior compartment.

7. The modular EEG cart of claim 6, further comprising a monitor coupled to the neck.

8. The modular EEG cart of claim 6, wherein the IT panel is mounted to the frame opposite the cable recess panel.

9. The modular EEG cart of claim 6, further comprising a base mounted to a bottom of the frame, the base coupled to a plurality of wheels.

10. The modular EEG cart of claim 6, further comprising a photic arm mounted to the frame.

11. The modular EEG cart of claim 6, further comprising a patient panel removably mounted to the frame opposite the technician panel, the patient panel having a tray disposed therein, the tray configured to support medical equipment for use in treating a patient.

12. The modular EEG cart of claim 11, wherein the patient panel further comprises a power supply connector for supporting medical equipment for use in treating the patient.

13. The modular EEG cart of claim 6, further comprising an IR light source mounted to the neck.

14. The modular EEG cart of claim 6, wherein each panel is constructed from non-porous, smooth materials resistant to staining and able to withstand frequent sanitization procedures.

15. The modular EEG cart of claim 6, wherein the modular EEG cart is constructed from medical-grade stainless steel, antimicrobial-coated plastics, or sealed composite materials that permit the use of hospital-grade disinfectants for effective cleaning.

16. The modular EEG cart of claim 6, further comprising a top surface mounted to the frame, the top surface configured to provide a surface to a technician using the modular EEG cart.

17. A modular EEG cart comprising:
a base;
a neck extending from the base, the neck having a hinge; and
a camera coupled to the neck opposite the base;
wherein the base is mounted on a plurality of wheels.

18. The modular EEG cart of claim 17, further comprising a photic arm mounted to the neck.

19. The modular EEG cart of claim 17, wherein the camera is mounted to the neck on a turntable camera mount configured to permit the camera to rotate.

20. The modular EEG cart of claim 17, further comprising a grounding chain attached to the base and configured to ground electronics mounted to the modular EEG cart.
